(19) United States
(12) Patent Application Publication
(10) Pub. No.: US 2019/0328474 A1
(43) Pub. Date: Oct. 31, 2019

(54) STEERABLE INTRODUCER FOR MINIMALLY INVASIVE SURGERY

(71) Applicant: KONINKLIJKE PHILIPS N.V., EINDHOVEN (NL)

(72) Inventors: Aleksandra POPOVIC, BOSTON, MA (US); David Paul NOONAN, NEW YORK, NY (US)

(21) Appl. No.: 16/309,135

(22) PCT Filed: Jun. 20, 2017
(86) PCT No.: PCT/EP2017/065124

§ 371 Date: Dec. 12, 2018

Related U.S. Application Data
(60) Provisional application No. 62/353,311, filed on Jun. 22, 2016.

(51) Int. Cl. A61B 34/00; A61B 90/14; A61B 34/10; A61B 90/00

(52) U.S. Cl. CPC A61B 34/70; A61B 90/14; A61B 34/10; A61B 90/361; A61B 2090/363; A61B 2034/107; A61B 2090/378; A61B 2090/373; A61B 90/37

(57) ABSTRACT

A system employing an articulated steerable introducer (40), an imaging controller (22) and a steerable introducer controller (41). The articulated steerable introducer (40) includes a plurality of linkages and one or more joints interconnecting the linkages. The imaging controller (22) controls a planning of a distal steering motion of the articulated steerable introducer (40) to a target position within an anatomical region. The steerable introducer controller (41) controls an actuation of the joint(s) to distally steer the articulated steerable introducer (40) to the target position within the anatomical region as planned by the imaging controller (22).

### FIELD OF THE INVENTION

The present disclosure generally relates to introducers for minimally invasive surgeries. The present disclosure specifically relates to an image guidance of steerable introducers into anatomical regions.

### BACKGROUND OF THE INVENTION

Introducers are used to provide an intervention instrument channel for minimally invasive “key-hole” surgery. An example of such introducers are neuro-introducers for endoscopic neurosurgery (e.g., ventriculetomy, tumor resection, shunt procedures) or trocars for surgery (e.g., cardiac, abdominal, lung, ENT).

Some of these introducers include a printed scale on a straight introducer sheath that is used to gauge a depth of instrument introduction into an anatomical region to thereby provide safe access of the anatomical region. For example, stereotactic frames or frameless navigation are implemented to assist a surgeon in controlling an insertion point and angle of insertion of the neuro-introducer at a desired depth.

One issue with introducers is straight introducer sheaths do not provide sufficient dexterity for obstacle avoidance. Additionally, the placement of the straight introducer sheaths is usually blind and not guided by live images of the anatomical region, which further decreases safety and increases risk of injury to important anatomical structures within the region.

### SUMMARY OF THE INVENTION

The present disclosure provides inventions utilizing an image guidance based placement control of numerous and various types of minimally invasive procedures incorporating a steerable introducer for providing an interventional instrument tunnel into an anatomical region (e.g., a thoracic region, a cranial region, an abdominal region, a dorsal region or a lumbar region).

One form of the inventions of the present disclosure is a system employing an articulated steerable introducer, an imaging controller and a steerable introducer controller. The articulated steerable introducer includes a plurality of linkages and one or more joints interconnecting the linkages. The imaging controller controls a planning of a distal steering motion of the articulated steerable introducer to a target position within an anatomical region. The steerable introducer controller controls an actuation of the joint(s) to distally steer the articulated steerable introducer to the target position within the anatomical region as planned by the imaging controller.

A second form of the inventions of the present disclosure is a method for placing an articulated steerable introducer within an anatomical region, the articulated steerable introducer including a plurality of linkages and one or more joints interconnecting the linkages. The method involves an imaging controller controlling a planning of a distal steering motion of an articulated steerable introducer to a target position within the anatomical region. The method further involves a steerable introducer controller controlling an actuation of the joint(s) to distally steer the articulated steerable introducer to the target position within the anatomical region as planned by the imaging controller.

For purposes of the inventions of the present disclosure, terms of the art including, but not limited to, “introducer”, “image registration”, “imaging modality”, “planning image”, “treatment image”, “stereotactic frame”, “fiducial markers”, “interventional instrument”, “path planner”, “image register”, “signal driver” and “visual servo” are to be interpreted as understood in the art of the present disclosure and as exemplary described herein.

For purposes of the inventions of the term “planned introducer path” broadly encompasses, as understood in the art of the present disclosure and exemplary described herein, a straight line segment for inserting an articulated steerable introducer to a placement position within an anatomical region, and a steering motion segment for distally steering articulated steerable introducer from a placement positon to a target position within anatomical region.

For purposes of the inventions of the present disclosure, the term “articulated steerable introducer” broadly encompasses any introducer structurally configured, entirely or partially, with motorized control of one or more joints (e.g., a pivot joint) serially connected with rigid linkages including a proximal linkage, a distal linkage and optionally one or more intermediate linkages.

For purposes of the present disclosure, the term “controller” broadly encompasses all structural configurations of an application specific main board or an application specific integrated circuit housed within or linked to a workstation for controlling an application of various inventive principles of the present disclosure as subsequently described herein. The structural configuration of the controller may include, but is not limited to, processor(s), computer-usable/computer readable storage medium(s), an operating system, application module(s), peripheral device controller(s), slot(s) and port(s).

For purposes of the present disclosure, the labels “introducer”, “planning imaging”, and “treatment imaging” used herein for the term “controller” distinguishes for identification purposes a particular controller from other controllers as described and claimed herein without specifying or implying any additional limitation to the term “controller”.

For purposes of the inventions of the present disclosure, the term “workstation” is to be broadly interpreted as understood in the art of the present disclosure and as exemplary described herein. Examples of a “workstation” include, but are not limited to, an assembly of one or more computing devices, a display/monitor, and one or more input devices (e.g., a keyboard, joysticks and mouse) in the form of a standalone computing system, a client computer, a desktop or a tablet.

For purposes of the present disclosure, the term “application module” broadly encompasses a module incorporated within or accessible by a controller consisting of an electronic circuit and/or an executable program (e.g., executable software stored on non-transitory computer readable medium(s) and/firmware) for executing a specific application.

The foregoing forms and other forms of the present disclosure as well as various features and advantages of the present disclosure will become further apparent from the following detailed description of various embodiments of the present disclosure read in conjunction with the accompanying drawings. The detailed description and drawings are merely illustrative of the present disclosure rather than limiting, the scope of the present disclosure being defined by the appended claims and equivalents thereof.

### BRIEF DESCRIPTION OF THE DRAWINGS

FIG. 1 illustrates an exemplary embodiment of a minimally invasive transcranial endoscopic neurosurgery in accordance with the inventive principles of the present disclosure.

FIGS. 2A-2C illustrate exemplary embodiments of workstations in accordance with the inventive principles of the present disclosure.

FIGS. 3A-3C illustrate an exemplary embodiment of an articulated steerable introducer having two (2) interconnected linkages in accordance with the inventive principles of the present disclosure.

FIGS. 4A-4F illustrate an exemplary embodiment of an articulated steerable introducer having three (3) interconnected linkages in accordance with the inventive principles of the present disclosure.

FIGS. 5A and 5B illustrate exemplary embodiments of channels within an articulated steerable introducer in accordance with the inventive principles of the present disclosure.

FIG. 6 illustrates a flowchart representative of an exemplary embodiment of an steerable introducer placement method in accordance with the inventive principles of the present disclosure.

FIGS. 7A-7D illustrate an exemplary execution of the flowchart illustrated in FIG. 6.

### DETAILED DESCRIPTION OF THE PREFERRED EMBODIMENTS

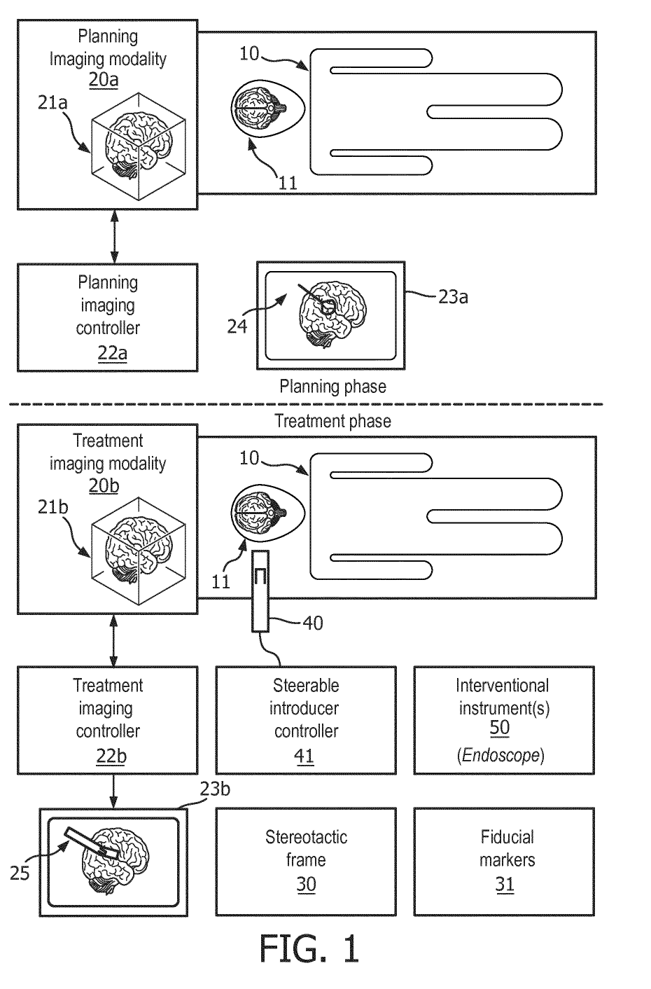

To facilitate an understanding of the present disclosure, the following description of FIG. 1 teaches basic inventive principles of an image guidance based placement control of an articulated steerable introducer 40 for facilitating performance of a minimally invasive transcranial endoscopic neurosurgery of a patient 10. From the description of FIG. 1, those having ordinary skill in the art will appreciate how to apply the inventive principles of the present disclosure to an image guidance based placement control of numerous and various types of minimally invasive procedures incorporating a steerable introducer for providing an interventional instrument tunnel into an anatomical region (e.g., a thoracic region, a cranial region, an abdominal region, a dorsal region or a lumbar region).

Referring to FIG. 1, a planning phase of the minimally invasive transcranial endoscopic neurosurgery involves a planning imaging controller 22a controlling a generation by a planning imaging modality 20a (e.g., a computed-tomography, a magnetic resonance, X-ray or an ultrasound imaging modality) as known in the art of a three-dimensional (“3D”) planning image 21a illustrative of a brain 11 within a cranial region of patient 10. During the planning phase, planning imaging controller 22a further controls a display as known in the art of planning image 21a of brain 11 on a monitor 23a for planning purposes, particularly for delineating a planned introducer path 24 traversing brain 11 within planning image 21a to thereby provide the interventional instrument tunnel into the cranial region of patient 10. Planned introducer path 24 includes a straight line segment for inserting an articulated steerable introducer 40 to a placement position within the cranial region of patient 10. Planned path 24 further includes one or more steering segments for distally steering introducer 40 in a pitch motion and/or a yaw motion to a target position within brain 11 of patient 10.

Still referring to FIG. 1, a treatment phase of the minimally invasive transcranial endoscopic neurosurgery initially involves a treatment imaging controller 22b controlling a generation by a treatment imaging modality 20b (e.g., a computed-tomography, a magnetic resonance, X-ray or an ultrasound imaging modality) as known in the art of two-dimensional (“2D”) treatment image(s) or 3D treatment image(s) 21b as shown for registration purposes.

For all embodiments, one or more treatment images 21b are registered to planning image 21a as known in the art.

In a stereotactic frame registration embodiment, a registration of a stereotactic frame 30 to planning image 21a as known in the art involves a generation of the treatment image 21b illustrative of stereotactic frame 30 affixed to a head and/or a neck of patient 10, or alternatively illustrative of a marker placement of a subsequent affixation of stereotactic frame 30 to the head and/or the neck of patient 10. The registration of stereotactic frame 30 to planning image 21a is accomplished by one of the controllers 22a and 22b in accordance with the following equation [1]:

^{DI}T_{SF}=^{TI}T_{SF}*^{PI}T_{TI} [1]

where ^{TI}T_{SF} is the transformation of stereotactic frame 30 to treatment image 21b,

where ^{PI}T_{H} is the transformation of treatment image 21b to planning image 21a, and

where ^{PI}T_{SF} is the transformation of stereotactic frame 30 to planning image 21a.

In a frameless stereotactic registration embodiment, a registration of a fiducial markers 31 to planning image 21a as known in the art involves a generation of a treatment image 21b illustrative of fiducial markers 31 affixed to a head of patient 10. The registration of fiducial markers 31 to planning image 21a is accomplished by one of the controllers 22a and 22b in accordance with the following equation [2]:

^{DI}T_{FM}=^{TI}T_{FM}*^{PI}T_{TI} [2]

where ^{TI}T_{FM} is the transformation of fiducial markers 31 to treatment image 21b,

where ^{PI}T_{H} is the transformation of treatment image 21b to planning image 21a, and

where ^{PI}T_{FM} is the transformation of fiducial markers 31 to planning image 21a.

Still referring to FIG. 1, subsequent to the image registrations, the treatment phase of the minimally invasive transcranial endoscopic neurosurgery further involves a minimal drilling of an entry point (not shown) into the cranial region of patient 10 whereby introducer 40 is inserted via imaging guidance into the cranial region along the straight segment of the registered planned introducer path 24 traversing brain 11 within planning image 21a.

In a treatment image insertion embodiment, a surgeon inserts introducer 40 into the entry point while viewing a treatment image 21b illustrative of an insertion of introducer 40 through the entry point into the cranial region of patient 10 and/or viewing an overlay of introducer 40 on the registered planning image 21a as introducer 40 is inserted through the entry point into brain 11 of patient 10.

In a stereotactic frame insertion embodiment, stereotactic frame 30 is affixed to the head and/or the neck of patient 10 as registered to planning image 21a, and adjusted to 3D coordinates of the entry point as illustrated in planning image 21a. The surgeon inserts introducer 40 via stereotactic frame 30 through the entry point into brain 11 of patient 10 as known in the art.

In a frameless stereotactic insertion embodiment, fiducial markers 31 are affixed to the head of patient 10 as registered to planning image 21a, and utilized to compute the 3D coordinates of the entry point as illustrated in planning image 21a. The surgeon inserts introducer 40 via stereotactic frame 30 through the computed entry point into brain 11 of patient 10 as known in the art.

Still referring to FIG. 1, subsequent to the straight-line insertion of introducer 40 into brain 11 of patient 10, the treatment phase of the minimally invasive transcranial endoscopic neurosurgery further involves a distal steering of introducer 40 along the pitch segment and/or the yaw segment of the registered planning image 21a to the target position within brain 11 of patient 10.

In a treatment image steering embodiment, a steerable introducer controller 41 executes a visual servo control of the distal steering of introducer 40 along the pitch segment and/or the yaw segment of the registered planning image 21a to the target position within brain 11 of patient 10 as will be further described herein.

In a stereotactic frame/frameless stereotactic steering embodiments, steerable introducer controller 41 executes an autonomous driving control of the distal steering of introducer 40 along the pitch segment and/or the yaw segment of the registered planning image 21a to the target position within brain 11 of patient 10 as will be further described herein.

Still referring to FIG. 1, subsequent to the distal steering of introducer 40 to the target position within brain 11 of patient 10, the treatment phase of the minimally invasive transcranial endoscopic neurosurgery finally involves a passing interventional instrument(s) 50 of any type through the interventional instrument tunnel established by the previous straight-line insertion and distal steering of introducer 40 to the target position within brain 11 of patient 10.

In practice, planning imaging modality 20a and treatment imaging modality 20b may or may not be the same type of imaging modality, or may be the same imaging modality

Also in practice, treatment imaging modality 20b may be operated to image the stereotactic frame/frameless stereotactic insertion and distal steering of the minimally invasive transcranial endoscopic neurosurgery.

Further in practice, the controllers of FIG. 1 may be installed within a single workstation or distributed across multiple workstations.

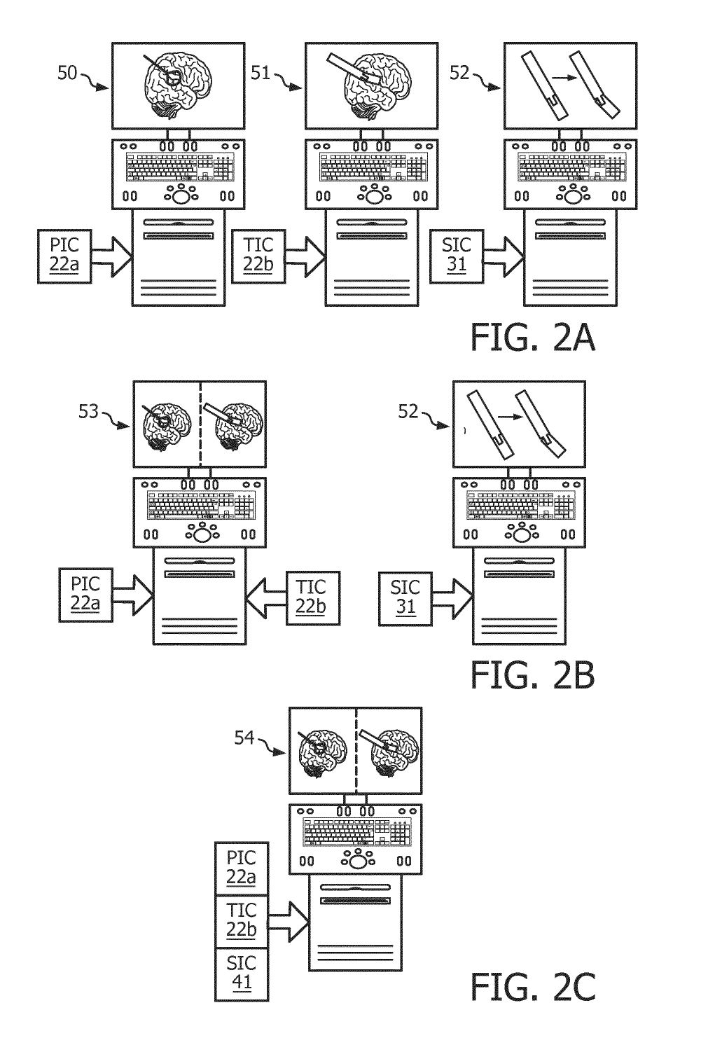

For example, FIG. 2A illustrates a planning imaging workstation 50 having planning imaging controller 22a installed therein for CT, MRI, X-ray or US imaging, and an treatment imaging workstation 51 having treatment imaging controller 22b installed therein for X-ray or US imaging. FIG. 2A further illustrates a steerable introducer workstation 52 having steerable introducer controller 41 installed therein for executing the visual servo control, the autonomous driving control or any other control technique for distally steering introducer 40.

Also by example, FIG. 2B illustrates an imaging workstation 53 having both planning imaging controller 22a and treatment imaging controller 22b installed therein for the same type of imaging or different types of imaging.

By further example, FIG. 2C illustrates an interventional workstation 54 having all controllers of FIG. 1 installed therein for the same type of imaging or different types of imaging and for executing the visual servo control, the autonomous driving control or any other control technique for distally steering introducer 40.

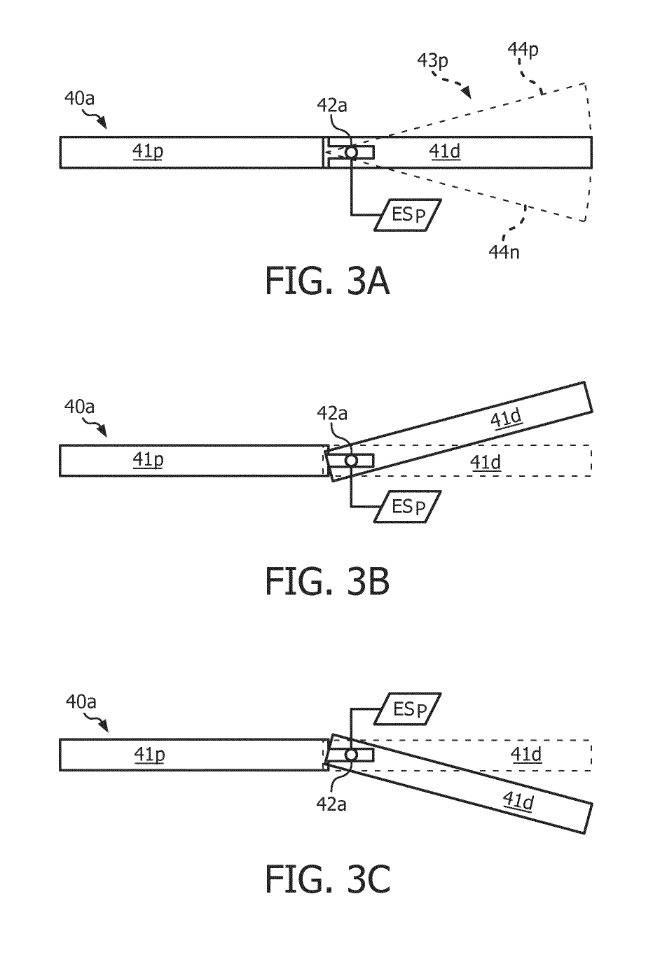

To facilitate a further understanding of the present disclosure, the following description of FIGS. 3-5 teaches basic inventive principles of an articulated steerable introducer. From this description, those having ordinary skill in the art will appreciate how to apply the inventive principles of the present disclosure to any type of articulated steerable introducer suitable for a minimally invasive procedure.

Generally, an articulated steerable introducer of the present disclosure employs a proximal linkage, a distal linkage and optionally one or more intermediate linkages. The articulated steerable introducer further includes joint(s) interconnecting the linkages in a complete or partial serial arrangement, and each pivot joint is controllable by a steerable introducer controller of the present disclosure.

In practice, a joint may be of any type of joint as known in the art including, but not limited to, a translational joint, a ball and socket joint, a hinge joint, a condyloid joint, a saddle joint and a rotary joint.

Also in practice, each pivot joint may be equipped with a motor for controlling a pose of each linkage, and/or a position sensor of any type (e.g., an encoder) for generating pose data informative of a pose (i.e., orientation and/or location) of the distal linkage relative to the proximal linkage.

For example, referring to FIG. 3A, an articulated steerable introducer 40a employs a proximal linkage 41p and a distal linkage 41d interconnected by a motorized pivot joint 42a controllable by a steerable introducer controller of the present disclosure to actuate a pitch motion of distal linkage 41d within a pitch envelope 43p defined by a max positive pitch 44p and a max negative pitch 44n.

Motorized pivot joint 42a is equipped with a rotary encoder (not shown) to generate an encoded pitch signal ESP informative of a pitch orientation of distal link 41d within pitch envelope 43p relative to proximal linkage 41p.

FIG. 3B shows an exemplary steering of distal linkage 41d to max position pitch 44p, and FIG. 3C shows an exemplary steering of distal linkage to max negative pitch orientation 44n.

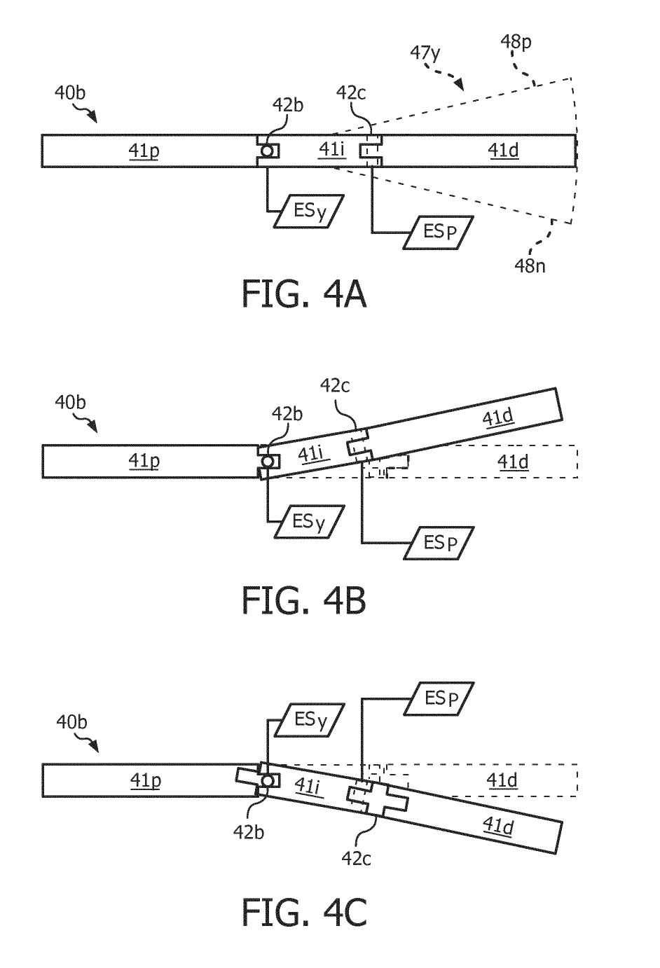

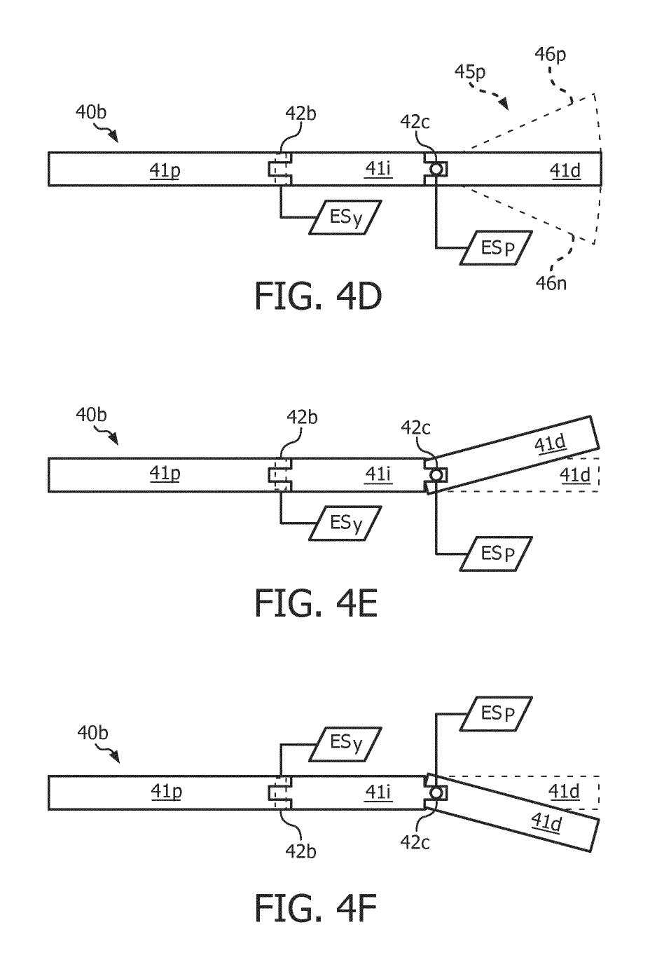

By further example, referring to FIG. 4A, an articulated steerable introducer 40b further employs proximal linkage 41p and an intermediate linkage 41i interconnected by a motorized pivot joint 42b controllable by a steerable introducer controller of the present disclosure to actuate a yaw motion of distal linkage 41d within a yaw envelope 47y defined by a max positive yaw 48p and a max negative yaw 48n.

Motorized pivot joint 42b is equipped with a rotary encoder (not shown) to generate an encoded pitch signal ES_{Y} informative of a yaw orientation of intermediate linkage 41i and distal linkage 41d within yaw envelope 47Y relative to proximal linkage 41p.

FIG. 4B shows an exemplary steering of intermediate linkage 41i and distal linkage 41d to max position yaw 48p, and FIG. 4C shows an exemplary steering of distal linkage to max negative yaw orientation 48n.

Referring to FIG. 4D, articulated steerable introducer 40b further employs intermediate linkage 41i and distal linkage 41d interconnected by a motorized pivot joint 42c controllable by a steerable introducer controller of the present disclosure to actuate a pitch motion of distal linkage 41d within a pitch envelope 45p defined by a max positive pitch 46p and a max negative pitch 46n.

Motorized pivot joint 42c is equipped with a rotary encoder (not shown) to generate an encoded pitch signal ESP informative of a pitch orientation of distal link 41d within pitch envelope 43p relative to intermediate linkage 41i.

FIG. 4E shows an exemplary steering of distal linkage 41d to max position pitch 46p, and FIG. 4F shows a steering of distal linkage to max negative pitch orientation 46n.

In practice, motorized pivot joints 42b and 42c may be adjacent or spaced as exemplary shown in FIGS. 4A-4F, or alternatively, intermediate linkage 41i may be omitted and motorized pivot joints 42b and 42c may be structurally integrated for actuating both a pitch motion and a yaw motion of distal linkage 41d.

Also in practice, a translational joint may interconnect proximal linkage 41p and intermediate linkage 41i to thereby translate intermediate linkage 41i and distal linkage 41d during an insertion of introducer 40b into an anatomical region.

Further in practice, the linkages of an articulated steerable introducer of the present disclosure may be structurally designed with one or more internal and/or external channels for interventional instruments.

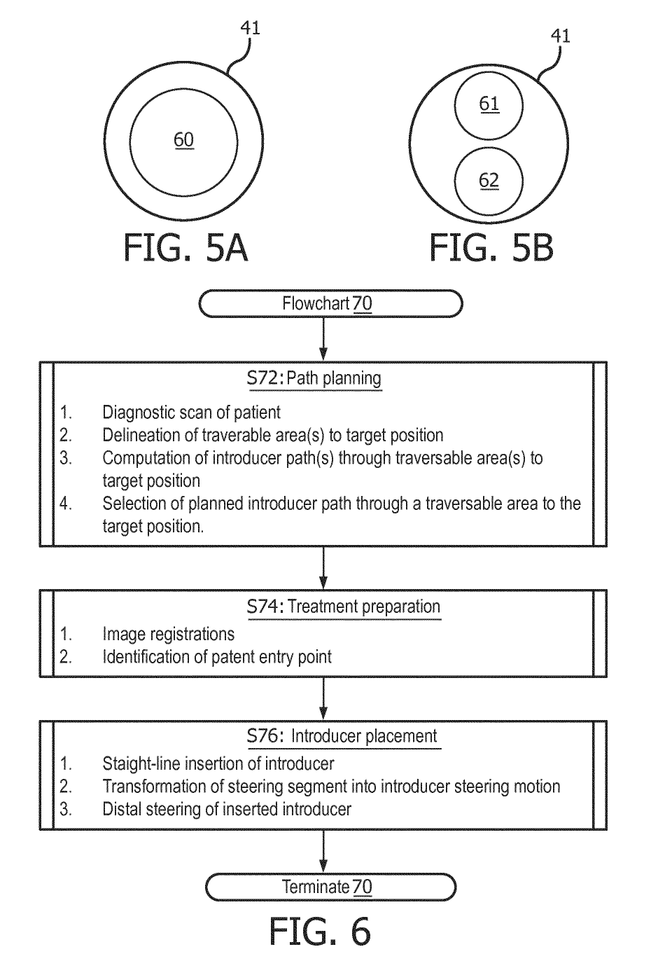

For example, FIG. 5A illustrates a single internal channel 60 extending through linkages 41 of articulated steerable introducer 40a shown in FIGS. 3A and 3B. For this channel embodiment, after placement of introducer 40a within an anatomical region, an endoscope may be first extended through channel 60 of linkages 41 and aligned with a distal tip of distal linkage 41d for imaging purposes. Thereafter, any additional interventional instruments may be extended through channel 60 of linkages as needed for surgical purposes.

By further example, FIG. 5B illustrates a pair of internal channels 61 and 62 extending through linkages 41 of articulated steerable introducer 40a shown in FIGS. 3A and 3B. For this channel embodiment, during the procedure, two (2) interventional instruments are simultaneously extended respectively through channels 61 and 62 for the aforementioned imaging and surgical purposes.

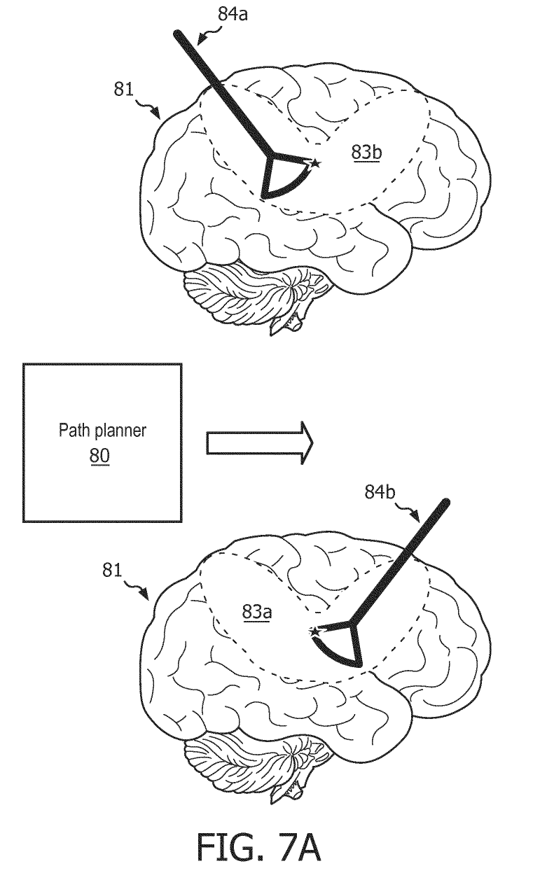

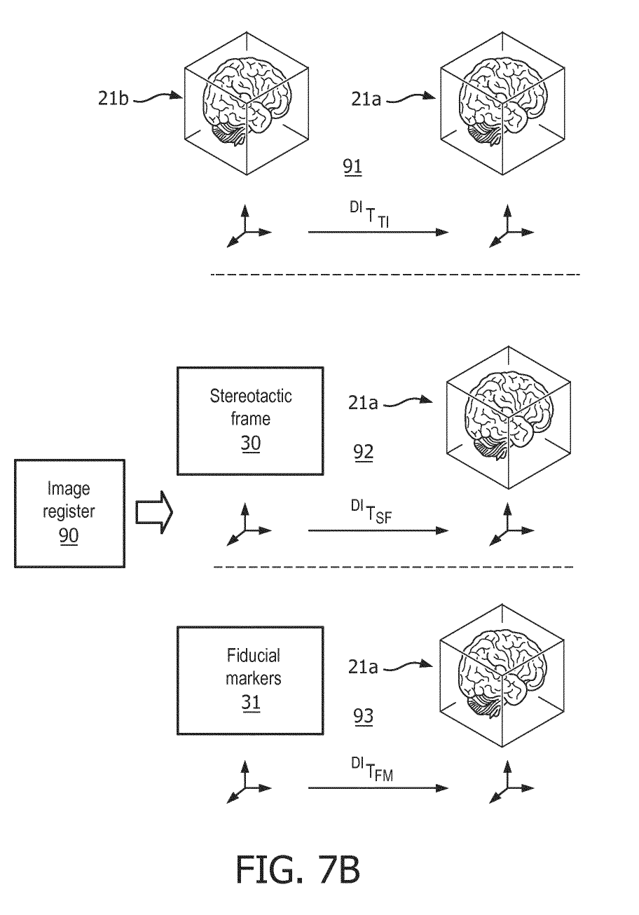

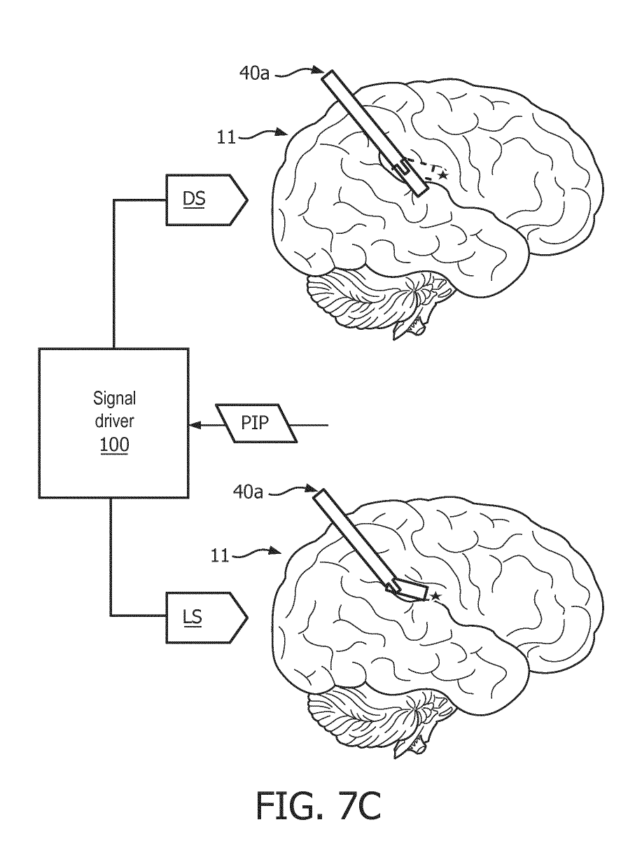

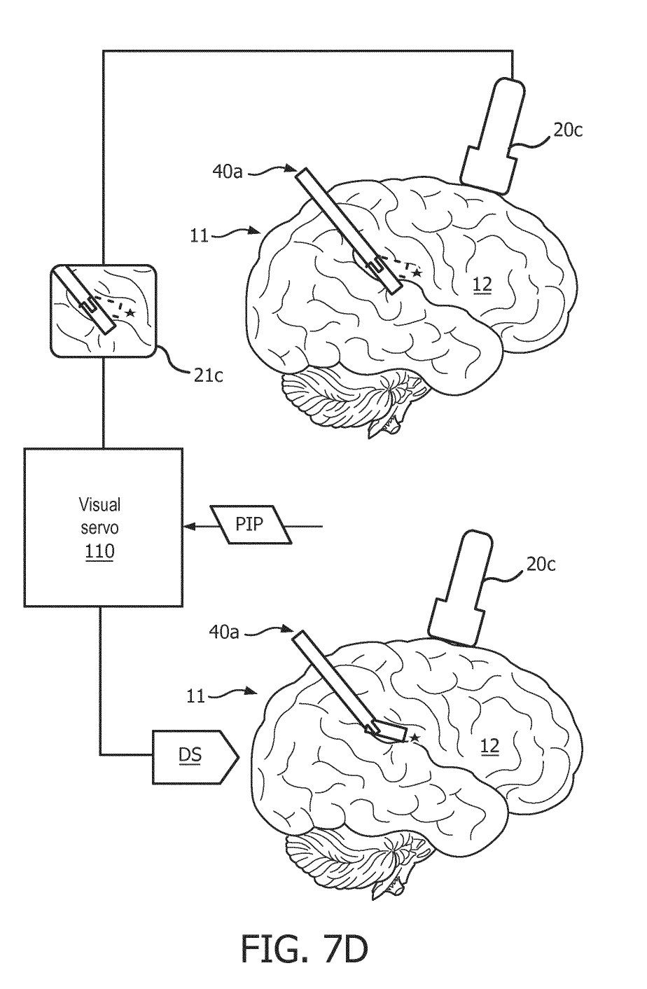

To facilitate a further understanding of the present disclosure, the following description of FIGS. 6 and 7 teaches basic inventive principles of steerable introducer placement methods of the present disclosure. From this description, those having ordinary skill in the art will appreciate how to apply the inventive principles of steerable introducer placement methods of the present disclosure for any type of minimally invasive procedure.

Referring to FIG. 6, a flowchart 70 representative of a steerable introducer placement method of the present disclosure involves a stage S72 for planning planning, a stage S74 for treatment preparation and a stage S76 for introducer placement.

Specifically, stage S72 of flowchart 70 encompasses (1) a planning scan of the patient, (2) a delineation of traversable area(s) by an introducer of the present disclosure an anatomical structure within the anatomical region to a target position, (3) a computation of all possible introducer path(s) through the traversable area(s) to the target position, and (4) a selection of a planned introducer path through a traversable area to the target position.

Each introducer path includes a straight line segment for inserting an introducer of the present disclosure to a placement position within the anatomical region. Each introducer path further includes a pitch segment and/or a yaw segment for distally steering an introducer of the present disclosure into the anatomical region to a target position within the anatomical region.

For example, FIG. 1 illustrates a planning image 21a of a brain 11 of a patient 10 generated by a planning imaging modality 20a as controlled by planning imaging controller 22a. For stage S72, the raw data of planning image 21a is loaded into a path planner 80 as shown in FIG. 7A whereby the raw data of planning image 21a is converted into a 3D working model 81 of brain 11.

Thereafter, a surgeon interfaces with path planner 80 to delineate the traversable areas, such as, for example, a traversable area 83a and a traversable area 83b through by working model 81 of brain 11 to a target position represented by a gray star as shown in FIG. 7A.

In practice, traversable areas will overlap around the target position as exemplary shown in FIG. 7A.

The surgeon further interfaces with path planner 80 to compute all possible introducer path(s) through the traversable area(s) to the target position, such as, for example, an introducer path 84a through traversable area 83a to the target position and an introducer path 84b through traversable area 83b to the target position as shown in FIG. 7A. Both introducer paths 84a and 84b include a straight line segment for inserting an introducer of the present disclosure to a placement position within the working model 81 of brain 11. Both introducer paths 84 and 84b further include a pitch segment for distally steering an introducer of the present disclosure to the target position in the working model 81 of brain 11.

The surgeon further interfaces with path planner 80 to select one of the computed introducer paths based on various factors relevant to the particular procedure including, but not limited to, an optimization to minimize distance between the introducer and sensitive structures, and an optimization to minimize the steering motion of the introducer.

In practice, path planner 80 may implement any virtual planning technique(s) known in the art that is suitable for the particular type of minimally invasive procedure being performed.

Also in practice, path planner 80 may be an application module of planning imaging controller 22a and/or treatment imaging controller 22b.

Referring back to FIG. 6, a stage S74 of flowchart 70 encompasses (1) an execution all necessary image registrations and (2) an identification on the patient of an entry point into the anatomical region.

For example, an image register 90 as shown in FIG. 7B is utilized to execute image registrations as needed including, but not limited to, an treatment image-planning image registration 91 involving a calculation of a transformation matrix ^{DI}T_{TI} as previously described herein, a calculation of a stereotactic frame-planning image registration 92 involving a calculation of a transformation matrix ^{DI}T_{SF} as previously described herein, and a calculation of a fiducial markers-planning image registration 92 involving a calculation of a transformation matrix ^{DI}T_{FM} as previously described herein.

The image registration(s) facilitates the identification on the patient of the entry point into the anatomical region as known in the art.

In practice, image register 90 may be implement any known transformation technique(s) as known in the art suitable for the particular type of minimally invasive procedure being performed.

Also in practice, image register 90 may be an application module of planning imaging controller 22a and/or treatment imaging controller 22b.

Referring back to FIG. 6, a stage S76 of flowchart 70 encompasses (1) a straight-line insertion of the introducer of the present disclosure and (2) a transformation of a steering segment of the planned introducer path into an introducer steering motion for (3) a distal steering the introducer of the present disclosure.

For example, as shown in FIG. 7C for stereotactic embodiments, introducer 40a (FIGS. 3A-3C) aligned in a straight configuration is introduced into brain 11 as known in the art. Upon completion of the insertion, a signal driver 100 transforms a steering segment of a planned introducer path PIP into an introducer steering motion via inverse kinematics of introducer 40a as known in the art whereby signal driver 100 initiates an application of a drive signal DS to motorized joints of introducer 40a to commences a distal steering of introducer 40a in accordance with the steering segment. Based on pose information provided to signal driver 100 via encoders of the motorized joints, signal driver 100 continues to apply drive signal DS until such time introducer 40a reaches the target position in accordance with the steering segment whereby signal driver 100 alternatively applies a locking signal LS to hold introducer 40a in the steered orientation.

In practice, signal driver 100 is an application module of steerable introducer controller 41.

Also by example, as shown in FIG. 7D for non-stereotactic embodiments, introducer 40a aligned in a straight configuration is introduced into brain 11 under the image guidance of an ultrasound prober 20c generating an ultrasound image 21c illustrative of introducer 40a within brain 11. Upon completion of the insertion as indicated by registered planning image 21a (not shown), a visual servo 110 transforms a steering segment of a planned introducer path PIP into an introducer steering motion by identifying the target position in ultrasound image 21c, determining a pitch direction of introducer 40a to the imaged target position in conformity with the steering segment and applies inverse kinematics as known in the art to application a drive signal DS to the motorized joints of introducer 40a until introducer 40a. This identification-determination-kinematics cycle is repeated by visual servo 110 for each acquisition of ultrasound image 21c until introducer 40a reaches the target position whereby visual servo 110 locks introducer 40a into the steering orientation.

In practice, visual servo 110 is an application module of steerable introducer controller 41.

Referring back to FIG. 6, flowchart 70 is terminated upon a completion of the introducer placement, or any of the stages S72-76 may be repeated to any degree as necessary.

Referring to FIGS. 1-9, those having ordinary skill in the art will appreciate numerous benefits of the present disclosure including, but not limited to, an image guidance control of a steerable introducer to a target position within an anatomical region in a safe manner that avoids obstacles.

Furthermore, as one having ordinary skill in the art will appreciate in view of the teachings provided herein, features, elements, components, etc. described in the present disclosure/specification and/or depicted in the drawings may be implemented in various combinations of electronic components/circuitry, hardware, executable software and executable firmware and provide functions which may be combined in a single element or multiple elements. For example, the functions of the various features, elements, components, etc. shown/illustrated/depicted in the drawings can be provided through the use of dedicated hardware as well as hardware capable of executing software in association with appropriate software. When provided by a processor, the functions can be provided by a single dedicated processor, by a single shared processor, or by a plurality of individual processors, some of which can be shared and/or multiplexed. Moreover, explicit use of the term “processor” should not be construed to refer exclusively to hardware capable of executing software, and can implicitly include, without limitation, digital signal processor (“DSP”) hardware, memory (e.g., read only memory (“ROM”) for storing software, random access memory (“RAM”), non-volatile storage, etc.) and virtually any means and/or machine (including hardware, software, firmware, circuitry, combinations thereof, etc.) which is capable of (and/or configurable) to perform and/or control a process.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (e.g., any elements developed that can perform the same or substantially similar function, regardless of structure). Thus, for example, it will be appreciated by one having ordinary skill in the art in view of the teachings provided herein that any block diagrams presented herein can represent conceptual views of illustrative system components and/or circuitry embodying the principles of the invention. Similarly, one having ordinary skill in the art should appreciate in view of the teachings provided herein that any flow charts, flow diagrams and the like can represent various processes which can be substantially represented in computer readable storage media and so executed by a computer, processor or other device with processing capabilities, whether or not such computer or processor is explicitly shown.

Furthermore, exemplary embodiments of the present disclosure can take the form of a computer program product or application module accessible from a computer-usable and/or computer-readable storage medium providing program code and/or instructions for use by or in connection with, e.g., a computer or any instruction execution system. In accordance with the present disclosure, a computer-usable or computer readable storage medium can be any apparatus that can, e.g., include, store, communicate, propagate or transport the program for use by or in connection with the instruction execution system, apparatus or device. Such exemplary medium can be, e.g., an electronic, magnetic, optical, electromagnetic, infrared or semiconductor system (or apparatus or device) or a propagation medium. Examples of a computer-readable medium include, e.g., a semiconductor or solid state memory, magnetic tape, a removable computer diskette, a random access memory (RAM), a read-only memory (ROM), flash (drive), a rigid magnetic disk and an optical disk. Current examples of optical disks include compact disk-read only memory (CD-ROM), compact disk-read/write (CD-R/W) and DVD. Further, it should be understood that any new computer-readable medium which may hereafter be developed should also be considered as computer-readable medium as may be used or referred to in accordance with exemplary embodiments of the present disclosure and disclosure.

Having described preferred and exemplary embodiments of novel and inventive steerable introducers for minimally invasive procedures (which embodiments are intended to be illustrative and not limiting), it is noted that modifications and variations can be made by persons having ordinary skill in the art in light of the teachings provided herein, including the drawings. It is therefore to be understood that changes can be made in/to the preferred and exemplary embodiments of the present disclosure which are within the scope of the embodiments disclosed herein.

Moreover, it is contemplated that corresponding and/or related systems incorporating and/or implementing the device or such as may be used/implemented in a device in accordance with the present disclosure are also contemplated and considered to be within the scope of the present disclosure. Further, corresponding and/or related method for manufacturing and/or using a device and/or system in accordance with the present disclosure are also contemplated and considered to be within the scope of the present disclosure.

1. A system, comprising:
an articulated steerable introducer,
wherein the articulated steerable introducer includes a plurality of linkages and at least one joint interconnecting the plurality of linkages, and
wherein the plurality of linkages includes a proximal linkage and a distal linkage;
an imaging controller,
wherein the imaging controller is configured to control a planning of a distal steering motion of the distal linkage relative to the proximal linkage from a placement position to a target position within an anatomical region; and
a steerable introducer controller,
wherein the steerable introducer controller is configured in communication with the at least one joint and the imaging controller to control an actuation of the at least one joint to distally steer the distal linkage relative to the proximal linkage from the placement position to the target position within the anatomical region as planned by the imaging controller.

2. The system of claim 1,
wherein the at least one joint includes a pivot joint interconnecting the proximal linkage and the distal linkage.

3. The system of claim 2,
wherein the pivot joint generates pose data informative of a pose of the distal linkage relative to the proximal linkage; and
wherein the steerable introducer controller, responsive to a generation of the pose data by the pivot joint, controls an actuation of pivot joint to steer the distal linkage relative to the proximal linkage from the placement position to the target position within the anatomical region as planned by the imaging controller.

4. The system of claim 2, further comprising:
a treatment imaging modality,
wherein the steerable introducer controller, responsive to a generation by the treatment imaging modality of treatment image data illustrative of the articulated steerable introducer within the anatomical region, controls an actuation of the pivot joint to distally steer the distal linkage relative to the proximal linkage from the placement position to the target position within the anatomical region as planned by the imaging controller.

5. The system of claim 1,
wherein the plurality of linkages include an intermediate linkage between a proximal linkage and a distal linkage; and
wherein the at least one joint includes
a proximal pivot joint interconnecting the proximal linkage and the intermediate linkage, and
a distal pivot joint interconnecting the intermediate linkage and the distal linkage

6. The system of claim 5,
wherein the proximal pivot joint generates pose data informative of a pose of the intermediate linkage and the distal linkage relative to the proximal linkage; and
wherein the steerable introducer controller, responsive to a generation of the pose data by the proximal pivot joint, controls an actuation of the proximal pivot joint to steer the intermediate linkage and the distal linkage relative to the proximal linkage within the anatomical region as planned by the imaging controller.

7. The system of claim 5,
wherein the distal pivot joint generates pose data informative of a pose of the distal linkage relative to the intermediate linkage; and
wherein the steerable introducer controller, responsive to a generation of the pose data by the distal pivot joint, controls an actuation of the distal pivot joint to steer the distal linkage within the anatomical region as planned by the imaging controller.

8. The system of claim 5, further comprising:
a treatment imaging modality,
wherein the steerable introducer controller, responsive to a generation by the treatment imaging modality of treatment image data illustrative of the articulated steerable introducer within the anatomical region, controls an actuation of the proximal pivot joint to steer the intermediate linkage and the distal linkage within the anatomical region as planned by the imaging controller.

9. The system of claim 5, further comprising:
a treatment imaging modality,
wherein the steerable introducer controller, responsive to a generation by the treatment imaging modality of treatment image data illustrative of the articulated steerable introducer within the anatomical region, controls an actuation of the distal pivot joint to steer the distal linkage within the anatomical region as planned by the imaging controller.

10. The system of claim 1,
wherein the plurality of linkages include an intermediate linkage between the proximal linkage and the distal linkage; and
wherein the at least one joint includes a translational joint interconnecting the proximal linkage to the intermediate linkage.

11. The system of claim 10,
wherein the translational joint generates pose data informative of a pose of the intermediate linkage relative to the proximal linkage; and
wherein the steerable introducer controller, responsive to a generation of the pose data by the translation joint, controls an actuation of the translation joint to translate the intermediate linkage and the distal linkage within the anatomical region as planned by the imaging controller.

12. The system of claim 11, further comprising:
a treatment imaging modality,
wherein the steerable introducer controller, responsive to a generation by the treatment imaging modality of treatment image data illustrative of the articulated steerable introducer within the anatomical region, controls an actuation of the translation joint to translate the intermediate linkage and the distal linkage within the anatomical region as planned by the imaging controller.

13. The system of claim 1,
wherein the imaging controller controls a planning of an introducer path within a traversable area of the anatomical region;
wherein the introducer path includes
a straight-line segment to insert the distal linkage within the anatomical region to the placement position; and
a steering motion segment to steer the distal linkage relative to the proximal linkage from the placement position to the target position within the anatomical region.

14. The system of claim 1, further comprising:
an ultrasound probe,
wherein the steerable introducer controller, responsive to a generation by the ultrasound probe of ultrasound image data illustrative of the articulated steerable introducer within the anatomical region, controls an actuation of the at least one joint to steer the distal linkage relative to the proximal linkage from the placement position within the anatomical region as planned by the imaging controller.

15. The system of claim 1, wherein the steerable introducer controller controls at least one of a signal driving actuation and a visual servo actuation of the at least one joint to distally steer the distal linkage relative to the proximal linkage from the placement position to the target position within the anatomical region as planned by the imaging controller.

16. A method for placing an articulated steerable introducer within an anatomical region, the articulated steerable introducer including a plurality of linkages and at least one joint interconnecting the plurality of linkages, the plurality of linkages including a proximal linkage and a distal linkage
the method comprising:
an imaging controller controlling a planning of a distal steering motion of the distal linkage relative to the proximal linkage from a placement position to a target position within the anatomical region; and
a steerable introducer controller controlling an actuation of the least one joint to distally steer the distal linkage relative to the proximal linkage from the placement position to the target position within the anatomical region as planned by the imaging controller.

17. The method of claim 16,
wherein the at least one joint includes a pivot joint between the proximal linkage and the distal linkage;
wherein the pivot joint generates pose data informative of a pose of the distal linkage relative to the proximal linkage; and
wherein the steerable introducer controller, responsive to a generation of the pose data by the pivot joint, controls an actuation of the pivot joint to steer the distal linkage from the placement position to the target position within the anatomical region as planned by the imaging controller.

18. The method of claim 16, further comprising:
a treatment imaging modality generating treatment image data illustrative of the articulated steerable introducer within the anatomical region;
wherein the at least one joint includes a pivot joint between the proximal linkage and the distal linkage; and
wherein the steerable introducer controller, responsive to the generation by the treatment imaging modality of the treatment image data, controls an actuation of the pivot joint to distally steer the distal linkage from the placement position to the target position within the anatomical region as planned by the imaging controller.

19. The method of claim 16,
wherein the imaging controller controls a planning of an introducer path within a traversable area of the anatomical region;
wherein the introducer path includes
a straight-line segment to insert the distal linkage within the anatomical region to the placement position; and
a steering motion segment to steer the distal linkage relative to the proximal linkage from the placement position to the target position within the anatomical region.

20. The method of claim 16,
wherein the steerable introducer controller controls at least one of a signal driving actuation and a visual servo actuation of the at least one joint to distally steer the distal linkage relative to the proximal linkage from the placement position to the target position within the anatomical region as planned by the imaging controller.
